(19) United States
(12) Patent Application Publication
(10) Pub. No.: US 2006/0030732 A1
(43) Pub. Date: Feb. 9, 2006

(54) Process for cleaving thiazolidines

(75) Inventors: Dieter Heldmann, Munchen (DE); Juergen Stohrer, Pullach (DE)

Correspondence Address: BROOKS KUSHMAN P.C., 1000 TOWN CENTER, TWENTY-SECOND FLOOR, SOUTHFIELD, MI, 48075, US

(73) Assignee: Consortium fur elektrochemische Industrie GmbH, Munich (DE)

(21) Appl. No.: 11/190,232

(22) Filed: Jul. 27, 2005

(30) Foreign Application Priority Data
Aug. 5, 2004 (DE) 10 2004 038 146.1

(51) Int. Cl. C07C 319/12

(52) U.S. Cl. 562/557; 562/426

(57) ABSTRACT

Preparation of α-substituted cysteine derivatives takes place by acidic hydrolysis of thiazolidines, wherein the hydrolysis is effected in the presence of a further phase comprising an organic solvent and a vapor comprising water, acid, organic solvent and aldehyde is removed from the reaction mixture, or a vapor comprising water, acid and aldehyde is removed from the reaction mixture and the vapor or its condensate is subsequently contacted with a phase comprising an organic solvent. The process requires less hydrolysis time and allows for recycle and/or recovery of acid and by product aldehyde.

### BACKGROUND OF THE INVENTION

1. Field of the Invention

The invention relates to a process for cleaving thiazolidines. In particular, the process is suitable for the accelerated cleavage of 4-substituted thiazolidines and the isolation of the R²CHO aldehyde cleavage product in addition to a substituted cysteine derivative.

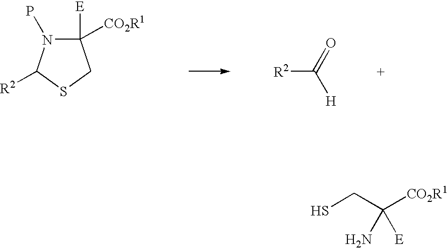

2. Description of the Related Art

The cleavage of thiazolidines by acids with very long reaction times is a known reaction for obtaining cysteine derivatives “incorporated” in the thiazolidines. See, for example G. Pattenden et al., TETRAHEDRON 1993, 49, 2131-2138. Since this acid cleavage is an equilibrium reaction, a shift in the equilibrium and thus complete reaction can be achieved by continuously removing the aldehyde from the equilibrium mixture. In the case of Pattenden et al., this is in all likelihood done by an acid-induced decomposition of the aldehyde, by oxidation of the aldehyde by traces of oxygen to pivalic acid, acid-induced trimerization, or by simple volatilization.

DE 10308580 discloses a process in which the aldehyde released in the hydrolysis is removed from the equilibrium selectively and continuously, alone or together with the acid used for the hydrolysis. It was possible to achieve a considerable acceleration of the hydrolysis from 3 d to 24-30 h by this method.

However, this process also has disadvantages. For instance, in order to remove the aldehyde formed from the reaction mixture very rapidly, a large amount of acid also has to be distilled off. The vapor withdrawn forms an aqueous distillate comprising acid and aldehyde. The acid is contaminated with the organic cleavage products and has to be discarded. The prolonged contact with acid results in the aldehyde entering into side reactions, which firstly forms crystalline trimer deposits which can block the condenser, and secondly forms decomposition products which cannot be characterized any further and do not allow viable reuse of the aldehyde on the basis of process technology and economic considerations.

### SUMMARY OF THE INVENTION

It is therefore an object of the invention to provide an alternative process which enables both rapid hydrolysis of thiazolidines and also provides for the recovery of the aldehyde withdrawn from the equilibrium mixture. These and other objects are achieved by a process in which the acidic hydrolysis is carried out at the boiling point of the reaction mixture in the presence of a further phase comprising an organic solvent, or in which the vapor withdrawn from the hydrolysis is additionally contacted with such an organic phase.

### DETAILED DESCRIPTION OF THE PREFERRED EMBODIMENT(S)

The invention provides a process for preparing compounds of the general formula (1) 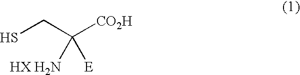
where
E is a linear or branched C₁-C₁₂-alkyl, C₃-C₁₀-alkenyl, C₆-C₁₅-aryl or C₇-C₂₁-aralkyl radical optionally substituted by halogen-, cyano-, nitro- or ester groups, and
X is halide, hydrogensulfate, dihydrogenphosphate, perchlorate, alkylsulfonate, arylsulfonate or fluoroalkylsulfonate,
by acidic hydrolysis of a compound of the general formula (2) 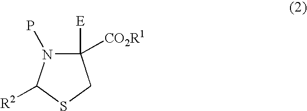
   where
R¹ is selected from the group consisting of hydrogen, alkali metals, alkaline earth metals, linear and branched C₁-C₁₂-alkyl, C₆-C₁₅-aryl and C₇-C₂₁-aralkyl radicals, dialkylsilyl, trialkylsilyl, dialkylarylsilyl, diarylalkylsilyl, and triarylsilyl radicals, wherein the alkyl radicals on the silyl radicals may are C₁-C₁₂-alkyl radicals and the aryl radicals on the silyl radicals are C₆-C₁₅-aryl radicals, and
R² is selected from the group consisting of linear and branched C₁-C₁₂-alkyl, C₆-C₁₅-aryl, and C₇-C₂₁-aralkyl radicals, and
P is an amino protecting group or hydrogen, and
comprising hydrolyzing with heating of the reaction mixture to boiling, and
effecting the hydrolysis in the presence of a further phase comprising an organic solvent and removing from the reaction mixture a vapor comprising water, acid, organic solvent and aldehyde, or removing from the reaction mixture a vapor comprising water, acid and aldehyde, and subsequently contacting the vapor or its condensate with a phase comprising an organic solvent.

The additional organic solvent phase may be added to the reaction mixture before and/or during the hydrolysis (cleavage reaction), in which case the process is carried out in the form of an extractive distillation. Alternatively, the organic phase may be contacted with the gaseous or recondensed distillate stream. It is also possible to combine the two aforementioned alternatives.

A common characterizing feature of the process according to the invention is the presence of a further phase comprising an organic solvent, which serves to extract the aldehyde released in the hydrolysis from the aqueous solution into the organic phase. The condensate of the vapor removed from the reaction mixture, the product of the vapor contacted with an organic phase and/or the mixture of the condensate of the vapor contacted with an organic phase is subsequently subjected to a phase separation into organic and aqueous phases.

In a typical embodiment of the process according to the invention, the aqueous, acidic phase comprising a compound of the general formula (2) is heated to boiling and an organic solvent is added, so that the aqueous acidic distillate comprising the aldehyde R²CHO forms a biphasic mixture with the added organic solvent, and the aldehyde preferably accumulates in the organic phases.

It has surprisingly been found that, especially as a result of the addition of an organic solvent during the hydrolysis, a significant acceleration of the reaction, and also recovery of the aldehyde, is achievable. In addition, the inventive procedure allows the amount of acid used to be reduced distinctly.

The reaction time for the quantitative hydrolysis by the process according to the invention is typically only 12 to 15 h and thus about half of the reaction time which can be attained with the process disclosed in DE 10308580.

The vapor removed from the reaction mixture and/or its condensate which has been contacted with an organic phase is subjected to a phase separation into aqueous and organic phases. The organic solvent which has been added to the reaction mixture before and/or during the hydrolysis actually brings about an immediate extraction, so that the aldehyde, after phase separation into aqueous and organic phase, can be removed virtually quantitatively with the organic phase.

The optional additional step of recycling of the aqueous phase comprising the acid back to the reaction mixture, on completion of the phase separation, additionally succeeds in appreciably lowering acid consumption. The recycling of the acid which has been distilled off allows the jacket temperature of the reaction vessel to be increased during the combined hydrolysis reaction and extractive distillation without simultaneously excessively increasing the acid consumption as a result of the associated higher proportion of acid distilled off. This leads to a higher volume flow rate in the distillation overall, and thus ultimately to a more rapid discharge of the aldehyde to be removed from the equilibrium mixture.

In a preferred embodiment of the process according to the invention, the organic phase is fed to an optional step for the recovery of the aldehyde present therein. In a further preferred embodiment, the aqueous acid phase removed by extraction is reused as acid in the cleavage reaction. This can be done later in a new reaction batch or, more preferably, directly by recycling into the original reaction mixture.

The E radical preferably represents optionally halogen, cyano-, nitro- or ester-functionalized, linear or branched C₁-C₁₂-alkyl, in particular methyl, ethyl, propyl, butyl, pentyl, hexyl, heptyl, octyl, nonyl or isopropyl radicals; C₆-C₁₅-aryl radicals; C₇-C₂₁-arylalkyl radicals; in particular benzyl or C₃-C₁₀-alkenyl radical, in particular, allyl radicals. E is most preferably methyl.

The amino protecting group P may, in addition to hydrogen, be any protecting group which is used commonly for the protection of amino groups. For the process according to the invention, it is possible to use all common amino protecting groups, for example those which are currently known to those skilled in the art from P. J. Kocienski, PROTECTING GROUPS, Thieme Verlag, 1994, p. 185-243.

In a preferred embodiment of the process according to the invention, compounds of the general formula (1) which are protected by N-acyl, N-sulfonyl, N-sulfenyl, or N-silyl derivatives, or with N-alkyl groups, are used. Particularly preferred amino protecting groups P are formyl, acetyl, trifluoroacetyl, methoxycarbonyl, ethoxycarbonyl, tert-butoxycarbonyl, benzyloxycarbonyl, allyloxycarbonyl, benzyl, trityl, trialkylsilyl (in particular trimethylsilyl, triethylsilyl, triisopropylsilyl, and tert-butyldimethylsilyl), aryldialkylsilyl (in particular phenyldimethylsilyl), diarylalkylsilyl (in particular diphenylmethylsilyl), and triarylsilyl (in particular triphenylsilyl). Very particular preference is given to formyl and acetyl.

The radicals R¹ may be selected from a multitude of possibilities, so that a multitude of substance classes results. Possible substance classes are organic or silyl esters, free acids or mono- or dicarboxylates of the free acid with metals of the first or second main group. Preferred radicals for R¹ which may be used in the process according to the invention are hydrogen, lithium, sodium, potassium, magnesium and calcium.

Further preferred radicals R¹ are selected from methyl, ethyl, n-propyl, isopropyl, n-butyl, tert-butyl, phenyl or benzyl radicals, and from trimethylsilyl, triethylsilyl, tributylsilyl, dimethylsilyl, diphenyl, tert-butyldimethylsilyl, thexyldimethylsilyl, norbornyldimethylsilyl, dimethylphenylsilyl, diphenylmethylsilyl, and triphenylsilyl radicals.

In the case of diorganosilyl radicals, in particular dimethylsilyl and diphenylsilyl, compounds of the structures shown below result, and can be converted by the process according to the invention in an analogous manner and can thus be used as reactants in the sense of compounds of the general formula (2), in particular in an optically pure configuration. 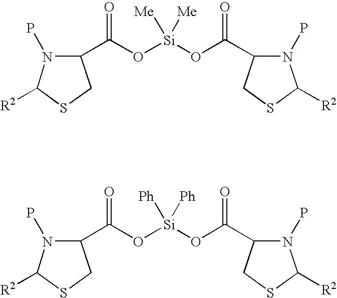
Particularly preferred radicals for R¹ are methyl or ethyl.

Preferred radicals for R² which can be used in the process according to the invention are methyl, ethyl, n-propyl, isopropyl, n-butyl, tert-butyl, cyclohexyl, phenyl, tolyl, naphthyl or benzyl. Particular preference is given to tert-butyl.

The process according to the invention can be used to prepare compounds of the general formula (1) in racemic form, and also for the preparation of optically pure isomers.

In one embodiment of the process according to the invention, compounds in the configuration of the formulae (1a) and (1b) 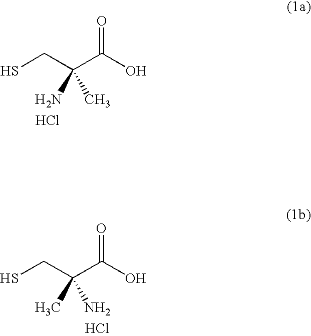
are obtained starting from the corresponding optically pure isomers of the general formulae (2a) and (2b) 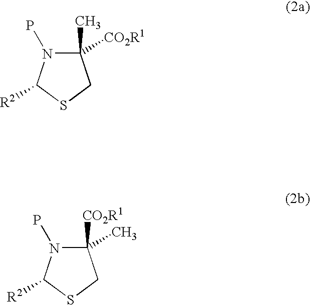
and subsequent inventive hydrolysis, where the R¹, R² and P radicals are each as defined above, and are in particular selected from their preferred embodiments.

A particularly preferred embodiment of the process according to the invention is the preparation of compounds of the general formula (1a) by inventive hydrolysis of compounds of the general formula (2c) 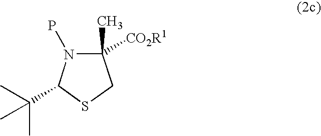
where
P and R¹ are each as defined above, and are in particular selected from their preferred embodiments. In this particularly preferred embodiment, it is found to be particularly advantageous that the hydrolysis is effected in the presence of a further phase comprising an organic solvent, and a vapor comprising water, acid, organic solvent and aldehyde is removed from the reaction mixture. In particular, the organic phase may be present from the start and will be metered in continuously during the hydrolysis. More preferably, P represents formyl and R¹ represents methyl. Optionally, the organic phase, on completion of phase separation of the vapor removed, may then be fed to the recovery of the pivalaldehyde present therein.

The process may be carried under different pressure conditions of from 10 mbar to 100 bar. Preference is given to carrying it out at from standard pressure to 5 bar gauge.

The concentration of the reactant of the general formula (2) in relation to the aqueous acid may be between 0.1 and 95% by weight; preference is given to establishing a maximum concentration of between 10 and 50% by weight.

Acids are understood quite generally to be Brönsted acids, in particular hydrochloric acid, sulfuric acid, nitric acid, hydrobromic acid, phosphoric acid, toluenesulfonic acid, methanesulfonic acid, oxalic acid or acetic acid. Particular preference is given to hydrochloric acid. In that case, Y in the general formula (1) consequently preferably represents chloride.

The reactant of the general formula (2) may optionally be fed to the aqueous hydrolysis solution as a solution in an organic solvent or as a solid. Suitable organic solvents as a portion or as the sole constituent of the organic phase for the process according to the invention are those organic solvents which can form a biphasic mixture with water or aqueous acids and are stable toward acid. Suitable solvents include esters such as ethyl acetate; ethers such as tetrahydrofuran, diethyl ether, and dibutyl ether; aliphatic C₅-C hydrocarbons, individually or as a mixture, such as pentane, hexane, cyclohexane, heptane, octane, nonane, decane, decahydronaphthalene and isomers thereof; higher alcohols (C₄-C₁₀) such as butanol, pentanol, hexanol, 2-ethylhexanol, heptanol, octanol, and phenol; and higher ketones (C₄-C₁₀) such as butanone, pentanone, hexanone, and isomers thereof.

Particularly preferred solvents are chlorinated aliphatic hydrocarbons, for example dichloromethane, chloroform, tetrachloromethane, chlorinated ethanes, ethenes, and propanes; aromatic C₆-C₁₅ hydrocarbons such as benzene, toluene, o-, m-, and p-xylene and mixed xylenes, mesitylene, diethylbenzene; chlorinated aromatic hydrocarbons such as chlorobenzene, dichlorobenzene, trichlorobenzene, tetrachlorobenzene, and chlorotoluenes.

Solvent which has been distilled off can be replaced during the reaction. The distillate which distills over is either collected and separated into the respective phases or passed through an organic phase comprising and/or consisting substantially of an organic solvent, and then separated into the respective phases. The last step is necessary when an organic phase was not already present in the reaction mixture and/or had not been added. The organic phase consists preferably of only one organic solvent.

On completion of phase separation, the removed aqueous acid phase is reused as acid in the cleavage reaction. This may be done later in a new reaction batch or, more preferably, directly by recycling into the original reaction mixture.

The organic phase which has been removed may be subjected to the recovery of the aldehyde present therein. This recovery may be effected by fractional distillation or by precipitation with reagents which form precipitates with carbonyls, in particular hydroxylamine, substituted phenylhydrazines or bisulfite adduct formation.

In an alternative embodiment of the process according to the invention, the hydrolysis reaction may also be carried out as what is known as a reactive distillation in a distillation column. Suitable column types are the embodiments which are customary on the industrial scale, for example columns having sieve trays, bubble-cap trays, random packings, which are equipped at different heights with feeds and draws for the reactant and the reaction products.

In this embodiment, the reactant of the general formula (2) is introduced as a mixture with the acid or the organic solvent at a suitable height (typically in the middle region) into the column, in which the aqueous acid used for the hydrolysis is present in gaseous and liquid (condensed) form. The main portion of the aqueous acid is disposed typically in the heated distillation still and is heated there in such a way that it does not reach the outlets of the column in the upper section of the column, i.e. is only kept at reflux in the lower and middle section of the column. The cleavage reaction (hydrolysis) then takes place in this region of the column. The low boilers formed in the cleavage reaction (in particular aldehyde, alcohols and/or organic acids) rise further within the column. In the upper section of the column, these low boilers are removed, optionally in a mixture with further cleavage products, the organic solvent, or small entrained residual amounts of the acid, via suitable draws at points of different height. Ideally, only pure products are removed at the draws at the points of different height in the column up to the top of the column.

The non-volatile cleavage product of the general formula (1) runs down the column and collects in the distillation still in the distillation bottom. As long as the reactant of the general formula (2) has not yet been fully converted to the target product of the general formula (1) here, the solution of the distillation still is fed back to the reactive distillation in the middle of the column. This operation is repeated until full conversion has been achieved.

The examples which follow serve to illustrate the invention in detail and are in no way to be interpreted as being limiting.

### EXAMPLE 1

### L-2-methylcysteine

A perforation apparatus (liquid/liquid extractor) was initially charged with methyl (2R,4R)-2-tert-butyl-3-formyl-1,3-thiazolidine-4-methyl-4-carboxylate (10.0 mg, 40.8 mmol) in dichloromethane (10 ml) and hydrochloric acid (20%, 20 ml). The reaction mixture was heated to reflux. The distillate which distilled over was condensed in an intensive cooler and passed through a dichloromethane phase. The hydrochloric acid which had distilled over was passed via an overflow back into the reaction flask. The reaction monitoring of the reaction mixture by NMR (D₂O) showed a conversion to L-2-methylcysteine of 99% after 10 h. The dichloromethane phase was worked up by distillation. 7.5 g of a colorless liquid were obtained. The content of pivalaldehyde in this dichloromethane solution by NMR is 45% (corresponds to 3.37 g of pivalaldehyde).

### Example 2

### L-2-methylcysteine

A 250 ml three-neck flask with two dropping funnels, distillation head with condenser and attached dropping funnel as a collecting vessel was initially charged with (2R,4R)-2-tert-butyl-3-formyl-1,3-thiazolidine-4-methyl-4-carboxylate (50.0 g, 216 mmol) in toluene (25 g), admixed with hydrochloric acid (20%, 100 g). The mixture was heated to boiling at a bath temperature of 130° C. The distillate was collected in a dropping funnel, discharged hourly and phase separated. The acid phase was recycled into the reaction flask via one dropping funnel. In addition, approx. 2-3 ml of toluene per hour were added to the reaction mixture. The individual toluene phases which had distilled over were removed and analyzed for the respective pivalaldehyde content. The following values were obtained (0.5 h, 44.7%), (1.5 h, 61.1%), (2.5 h, 33.2%), (4.5 h, 31.9%), (5.5 h, 3.8%), (6.5 h, 4.5%), (7.5 h, 1.9%), (8.5 h, 0.8%). The reaction mixture showed the following conversion to L 2-methylcysteine (4.0 h, 74%), (8.0 h, 96%). After 8.5 h, toluene (20 ml) was added once again to the reaction mixture and this amount was distilled out within 4 h. Afterward, the conversion to L-2-methylcysteine was >99%.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A process for preparing compounds of the formula (1) 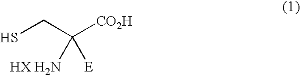
where
E comprises a linear or branched C₁-C₁₂-alkyl, C₃-C₁₀-alkenyl, C₆-C₁₅-aryl or C₇-C₂₁-aralkyl radical, each optionally substituted by one or more halogen-, cyano-, nitro- or ester groups, and
X is halide, hydrogensulfate, dihydrogenphosphate, perchlorate, alkylsulfonate, arylsulfonate or fluoroalkylsulfonate,
by hydrolyzing of a compound of the general formula (2) in the presence of acid 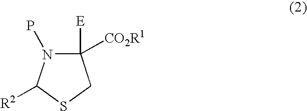
where
R¹ is selected from the group consisting of hydrogen, metals, alkali alkaline earth metals, linear and branched, optionally substituted C₁-C₁₂-alkyl, C₆-C₁₅-aryl and C₇-C₂₁-aralkyl radicals, dialkylsilyl, trialkylsilyl, dialkylarylsilyl, diarylalkylsilyl, and triarylsilyl radicals, the alkyl and aryl radicals on the silyl radicals selected from the group consisting of optionally substituted C₁-C₁₂-alkyl and optionally substituted C₆-C₁₅-aryl radicals;
R² is selected from the group consisting linear and branched, optionally substituted C₁-C₁₋₂-alkyl, C₆-C₁₅-aryl and C₇-C₂₁-aralkyl radicals, and
P is an amino protecting group or hydrogen, and
said hydrolyzing is carried out with heating of the reaction mixture to boiling, and effecting the hydrolysis a) in the presence of a further phase comprising an organic solvent and removing from the reaction mixture a vapor comprising water, acid, organic solvent and aldehyde, b) removing from the reaction mixture a vapor comprising water, acid and aldehyde, and subsequently contacting the vapor or its condensate with a phase comprising an organic solvent, or a combination of a) and b).

2. The process of claim 1, wherein a condensate of vapor removed from the reaction mixture, the product of vapor contacted with an organic phase, and/or a mixture of the condensate with the vapor contacted with an organic phase are subjected to a phase separation into organic and aqueous phases.

3. The process of claim 1, wherein the organic phase comprises one or more of the organic solvents tetrahydrofuran, diethyl ether, dibutyl ether, pentane, hexane, cyclohexane, heptane, octane, nonane, decane, decahydronaphthalene, butanol, pentanol, hexanol, 2-ethylhexanol, heptanol, octanol, phenol, butanone, pentanone, hexanone, dichloromethane, chloroform, tetrachloromethane, benzene, toluene, o-, m-, p-xylene, mesitylene, diethylbenzene, chlorobenzene, dichlorobenzene, trichlorobenzene, tetrachlorobenzene or chlorotoluene(s).

4. The process of claim 1, wherein, when hydrolysis takes place in the presence of an organic phase, the organic phase is added directly to the reaction mixture before and/or during the hydrolysis.

5. The process of claim 1, wherein the aldehyde present in the organic phase is recovered therefrom.

6. The process of claim 1, wherein the acid removed via the vapor from the reaction mixture is fed back to the reaction mixture.

7. The process of claim 1, wherein a compound of the formula (1a) 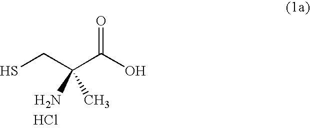
is prepared by acid hydrolysis of a compound of the formula (2c)

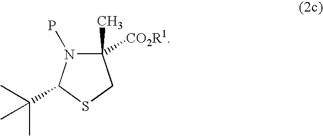

8. The process of claim 1, wherein the acidic hydrolysis is carried out in the presence of hydrochloric acid.

9. The process of claim 1, wherein the hydrolysis is carried out at from standard pressure to 5 bar gauge.

10. The process of claim 1, wherein the hydrolysis is carried out as a reactive distillation in a distillation column.
